(19) United States
(12) Patent Application Publication
(10) Pub. No.: US 2011/0270053 A1
(43) Pub. Date: Nov. 3, 2011

(54) Intra-Oral Detector and System for Modification of Undesired Behaviors and Methods Thereof

(75) Inventors: David Scott Utley, Redwood City, CA (US); Jack Denton Utley, Fairview, PA (US); Kathryn Mary Utley, Redwood City, CA (US); Alek Denton Utley, Redwood City, CA (US); Haley Grace Utley, Redwood City, CA (US); MacKenzie Taylor Utley, Redwood City, CA (US); Emma Kathryn Utley, Redwood City, CA (US)

(73) Assignee: David Scott Utley, Redwood City, CA (US)

(21) Appl. No.: 12/913,254

(22) Filed: Oct. 27, 2010

Related U.S. Application Data
(60) Provisional application No. 61/255,427, filed on Oct. 27, 2009.

(51) Int. Cl. A61B 5/00; A61B 5/08

(52) U.S. Cl. 600/309; 600/532

(57) ABSTRACT

Methods, devices and systems are disclosed for use in detecting a number of substances that may be present, whether constantly or intermittently, in the oral cavity and indicative of specific undesirable and/or unhealthy behaviors. Disclosed devices including detectors that indicate the presence of targeted substances qualitatively or quantitatively, may be secured temporarily, semi-permanently or permanently in the oral cavity employing known dental materials and techniques. The disclosed methods and devices enable constant passive monitoring and therefore may aid in the cessation via a number of positive and negative feedback mechanisms, as well as providing additional diagnostic information to healthcare providers.

### RELATED APPLICATION DATA

This application claims the benefit of priority of U.S. Provisional Patent Application Ser. No. 61/255,427 filed Oct. 27, 2009, and titled Intra-Oral Devices and Methods, which is incorporated by reference herein in its entirety.

### FIELD OF THE INVENTION

The present invention generally relates to the field of behavior modification and devices and systems thereof, and more particularly to apparatus and methods for assisting a user in cessation of undesired behaviors involving intake of substances through the oral pathway.

### BACKGROUND

Certain persons may engage in certain undesirable activities that have significant negative acute and/or long-term health, social, safety and financial implications for the person and for others associated with the person. In some cases, the person may wish to quit this behavior. Often, the person's family, friends, and/or healthcare providers are also engaging with the person in an attempt to have them quit the behavior. Often, such associated persons may also be negatively impacted by the behavior, socially, financially, and from a health perspective. Behavioral modification or cessation attempts to terminate the undesirable behavior may be formal and administered by healthcare professionals, or the person may take their own personalized approach to cessation. Either way, there is a role for a means to facilitate and optimize the success rate of the cessation process, as presently cessation programs have high acute failure rates and high recidivism rates.

A number of undesirable behaviors that involve smoking of one or more substances, oral ingestion of one or more substances, absorption via the gastrointestinal tract of one or more substances, oral placement of one or more substances, transdermal absorption of one or more substances, nasal sniffing of one or more substances, inhaling of one or more substances, intravenous injection of one or more substances, and other methods of absorbing one or more substances result in the subsequent presence of one or more of the substances or their metabolites in the exhaled breath, oral mucosa, and/or secreted saliva of the person either immediately or sometime after use of the substance. The presence of a unique substance or substances in the exhaled breath, oral mucosa, and/or secreted saliva can be detected in the intra-oral space and can then be used as a surrogate marker for the specific undesirable behavior. Passive and constant monitoring of the intra-oral space for presence of these surrogates would enable one to determine if the user was participating in the undesirable behavior, and could additionally quantify the amount of the behavior based on the amount of detected substance(s).

### SUMMARY OF THE DISCLOSURE

Exemplary embodiments of the present invention include a device or system capable of detecting substances within the oral cavity as an adjunct to achieving cessation of an undesirable behavior and/or maintaining abstinence after successfully quitting an undesirable behavior. Such embodiments may comprise at least one passive detector unit disposed in the oral cavity capable of detecting one or more substances indicative of the undesirable behavior, at least one anchoring means by which to secure the detector unit within the oral cavity, and, in some cases, a platform which interconnects the detector and anchor.

In various alternative embodiments, a detector is capable of detecting one or more substances of interest within inhaled gas and particulates, exhaled gas and particulates, intra-oral gasses (volatilized substances), ingested liquids and solids, intra-oral mucous, secreted saliva, and other means. Such a detector may be capable of detecting intra-oral substances indicative of smoking behavior including smoking of tobacco via cigarettes, pipes, cigars, and water pipes and smoking of illegal products such as marijuana, cocaine, heroin, and others. In other embodiments, the detector may be capable of detecting intra-oral substances indicative of the oral ingestion of certain substances, such as alcohol, caffeine-containing products, sugars, prescription drugs in excess, illegal drugs, and other substances. Alternative detectors may be capable of detecting intra-oral substances indicative of oral placement of certain substances, including but not limited to placing chewing tobacco and snuff in the oral cavity. Other detectors suitable for use in embodiments of the invention may be capable of detecting intra-oral substances indicative of transdermal absorption of certain substances, including but not limited to application on the skin of certain creams, ointments, gels, patches or other products that contain drugs of abuse, such as narcotics, LSD, steroids, and others. Still other detectors may be capable of detecting intra-oral substances indicative of nasal sniffing of drugs or substances of abuse, which include but are not limited to sniffing cocaine or sniffing glue, or capable of detecting intra-oral substances indicative of intravenous injection of drugs.

In other alternative embodiments the anchor means may comprise one or more bone screws inserted into the maxilla or mandible or both, a dental veneer affixed to one or more tooth surfaces, an orthodontic band or bracket, an orthodontic retainer, or other such device that is affixed to the dental enamel or encircled around one or more teeth, a retainer that is molded to fit over the dentition, such as a clear mouth guard or clear orthodontic retention device.

In exemplary embodiments the anchor means, detector unit, and platform may be distinct components or any two or more may be integrated into common components.

Exemplary embodiments of the invention may employ detector units that indicate the presence of a substance of interest qualitatively, such as by color change, or quantitatively. Such qualitative detectors may present a color change upon exposure to one or more substances of interest, with the following variables: color change can be either irreversible, spontaneously reversible, or reversible upon exposure to a certain specially designed medium such as a light, liquid or chemical; color change may occur upon exposure to any amount of the substance of interest (binary, positive or negative) or may have a pre-determine threshold at which the color change may occur which may range from very low levels of exposure to very high levels of exposure; color change may be variable in that more severe exposures are cue color or one color density, while other levels of exposure are a different color or a different color density; reversible colorimetric detectors may change color upon any preset level of exposure to a substance of interest, but then may return to the baseline level over a predetermined period of time as exposure to the substance is decreased or ceased; baseline color of colorimetric detector may be unobtrusive, such as white or clear or pink, and the detector may be worn in a location in the intra-oral space that is visible (upper incisors and canines) to the user and user's acquaintances, so that when the detector changes color the user and others will be aware that they are engaging in the undesirable behavior, and/or in non-reversible embodiments; the color change with additional exposure can be cumulative, therefore the amount of color change can be quantifiable and related to the amount of longitudinal exposure to the substance of interest. Quantitatively based detectors may be capable of quantifying the amount of substance exposure over time when removed and analyzed at periodic intervals during monitoring, such as every week.

Other exemplary embodiments of the invention include methods for facilitating cessation of an undesirable behavior, or ensuring prolonged abstinence from an undesirable behavior, by passively and continually monitoring for and detecting the presence of substances within the intra-oral space that are indicative of that undesirable behavior. Exemplary embodiments may comprise providing one or more intra-oral detectors that reside within the intra-oral cavity, retaining the detectors within the oral cavity, exchanging the detectors and other portions of the device as needed, and/or testing detectors for qualitative and/or quantifiable evidence of longitudinal exposure to substances of interest.

In further exemplary embodiments of the present invention, detectors as disclosed may be used within a formal behavior modification program, such as a smoking cessation program or an alcohol-dependency program, to facilitate cessation of an undesirable behavior. Such devices may be used independently by the user to facilitate a self-motivated behavior modification program, or as fixed by a health care professional if compliance is more problematic, to facilitate cessation of an undesirable behavior. As one further example, monitoring for a substance of interest according to embodiments of the present invention may be employed after successfully completing a cessation program to objectively confirm abstinence as well as to objectively monitor for recidivism.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a perspective view of a patient's maxilla (shown inverted for purposes of illustration) and a clear plastic retainer-type device including detector units according to an embodiment of the present invention configured to be received over the teeth of the maxilla.

FIG. 2 is an inside top view of a wire retainer-type device with a detector unit according to an embodiment of the present invention received over the teeth of the upper jaw.

FIG. 3 is a partial cross-sectional view of a tooth including an inlayed detector unit according to another embodiment of the present invention.

FIG. 4 is a front view of a patient's teeth including a plurality of porcelain veneer detector units according to an embodiment of the present invention.

FIG. 5 is a cross-sectional view of one of the teeth including the porcelain veneer detector unit as shown in FIG. 4.

FIG. 6 is a view of the embodiment of the invention in FIG. 4, illustrating an exemplary indication of the presence of a surrogate marker.

FIG. 7 is another view of an embodiment as in FIG. 4, illustrating an alternative exemplary indication of the presence of a surrogate marker.

FIGS. 8A and 8B are schematic side views of further alternative embodiments of the invention utilizing alternative anchor elements.

### DETAILED DESCRIPTION

Embodiments of the present invention comprise methods, devices and systems for use in detecting a number of substances that may be present, whether constantly or intermittently, in the oral cavity and indicative of specific undesired and/or unhealthy behaviors. Methods, devices and systems according to embodiments of the present invention enable constant passive monitoring for these substance and their related undesired behaviors, and therefore may aid in the cessation of said behaviors via a number of positive and negative feedback mechanisms, as well as via providing additional diagnostic information to the healthcare provider regarding response to therapy.

A variety of different behaviors may be targeted by embodiments of the present invention. Examples include smoking, oral ingestion, oral placement, transdermal absorption involving oral pathways, nasal inhalation, and/or intravenous injection of various substances. Some more specific examples of smoking behaviors include but are not limited to smoking of tobacco via cigarettes, pipes, cigars, and water pipes and smoking of illegal products such as marijuana, cocaine, heroin, and others. Oral ingestion may involve substances such as alcohol, caffeine-containing products, sugars, prescription drugs in excess, illegal drugs, and other substances. Oral placement may involve substances and behaviors such as placing chewing tobacco and snuff in the oral cavity. Transdermal absorption may involve application on the skin of certain creams, ointments, gels, patches or other products that contain drugs of abuse, such as narcotics, LSD, steroids, and others. Nasal inhalation is a common behavior commonly associated with substances of abuse, including but not limited to sniffing cocaine or glue products.

In general, embodiments of the present invention are configured to detect substances within the oral cavity that are surrogate markers for a behavior that is determined to be undesired or otherwise to be treated. Such marker substances may be present in the oral cavity due to one or more of the following distinct mechanisms:

1) In the case where a product is ignited and the subsequent smoke plume drawn into the mouth and lungs, the particulate and chemical matter containing the substance(s) of interest is deposited in part onto the oral mucosa and dentition, retained within the saliva and mucous, and deposited on or within other objects in the oral cavity.

2) In the case where a previously inhaled plume of smoke resulted in the substance being absorbed into the body and bloodstream via the lungs and then released from the bloodstream back into the pulmonary airspace for exhalation, the substance of interest is deposited in part onto the oral mucosa and dentition, retained within the saliva and mucous, and deposited on or within other objects in the oral cavity.

3) In the case of inhaling a plume of smoke, a portion of the plume enters only the pulmonary dead space (non-absorbing portion of the pulmonary air space) and is exhaled immediately through the oral cavity without absorption of the substance(s) into the bloodstream. In this case, the substance of interest is deposited in part onto the oral mucosa and dentition, retained within the saliva and mucous, and deposited on or within other objects in the oral cavity.

4) In the case where the substance is present in the oral cavity as a result of oral ingestion or oral placement of certain products containing the substance(s), the substance(s) of interest is deposited in part onto the oral mucosa and dentition and retained within the saliva and mucous as it is being swallowed or rinsed around in the mouth, and deposited on or within other objects in the oral cavity.

5) In the case where the substance was previously absorbed into the body and bloodstream via oral ingestion via the gastrointestinal system, oral placement, nasal sniffing, inhalation via the lungs, trans-dermal absorption, intravenous injection or other routes, the substance of interest is released from salivary glands into the oral cavity deposited in part onto the oral mucosa and dentition, retained within the saliva and mucous, and deposited on or within other objects in the oral cavity.

In general, devices according to embodiments of the present invention are placed and retained within the oral cavity of a user. The term “device” is used collectively herein to refer to the entire unit (or units) that is/are disposed within the oral cavity. As described in more detail herein below, exemplary embodiments of the present invention may have one or more of any of the following sub-units, such as: an anchoring component for affixing to a structure, a detector unit for passively monitoring for a substance of interest, and a platform upon which to interconnect the detector and anchoring component. The anchor device may be a bone screw, a wire, an orthodontic bracket, a dental veneer, an orthodontic band, a semi-permanently attached retainer, an arch wire, a palatal arch structure, a dental cap, a dental insert, a dental clasp, or other such means. When affixing an anchor to the dental enamel, various light-cured and non-light cured adhesives may be used or clasps with pressure fits may be used. The bone screws could be inserted in the mandible or maxilla in various locations. The platform and detectors may be attached to one or more anchors in the oral cavity and the latter may be positioned in one or more locations within the oral cavity.

Preferably, devices according to embodiments of the present invention are configured to substantially remain in the oral cavity during the period of monitoring for the substance that is the surrogate marker for the targeted behavior. In certain embodiments, the device will remain in the oral cavity for the complete monitoring interval. This may be accomplished voluntarily with a device that can be taken in and out of the oral cavity by the user, with the assumption that the user will comply and keep the device in place during the majority of the monitoring interval, or involuntarily with a device that is at least semi-permanently attached within the oral cavity and is not able to be removed by the user. In some embodiments, the user may be able to intermittently remove the device for cleaning and other processes. However, it is generally contemplated that embodiments that do not permit user removal, or at least do not facilitate user removal, will provide more optimal and constant monitoring for undesirable behaviors.

When disposed within the oral cavity, at least a portion of a device, specifically the detector portion, is exposed to the oral cavity milieu, which includes saliva, exhaled breath, inhaled substances, volatilized substances, food, liquids, etc. The detector portion of the device is therefore exposed to the substance of interest within the oral cavity via the multitude of mechanisms disclosed earlier in this patent application.

In some embodiments, the device may be placed in the oral cavity by the user without the need for a healthcare professional to semi-permanently affix the device. In this case, the user may exchange all or portions of the device (such as the detector sub-unit) as required at regular intervals and/or may be able to remove all or portions of the device intermittently to clean the oral cavity and the device. Such an embodiment may reduce the ability to monitor 100% of the time interval of interest for a user, however, with many of the substances of interest, prior behaviors can be detected in subsequent sampling of the oral cavity milieu, as in the example of carbon monoxide detection hours after smoking a cigarette. Therefore, intermittent removal of the device should not substantially limit the effectiveness of the device in monitoring for surrogate markers of undesirable behavior. Alternatively, or additionally, a separate detector unit may be included that is indicative of residence time in the oral cavity, for example by indicating a qualitative change correlated to an amount of exposure to naturally occurring substances in the saliva such as particular enzymes.

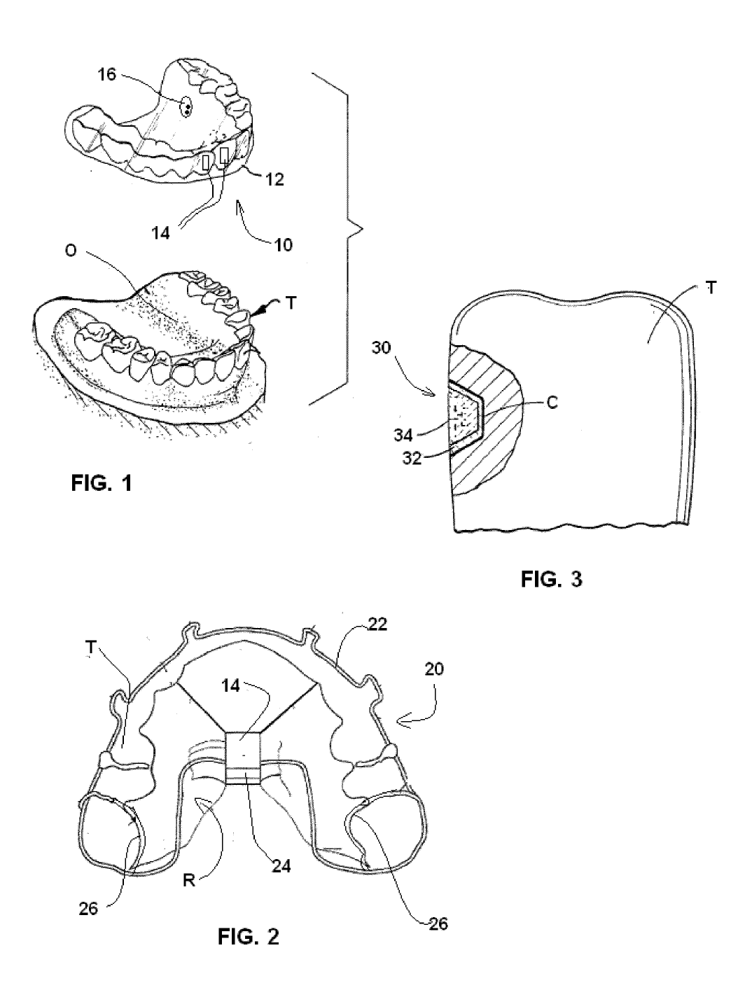

Referring now to FIG. 1, one exemplary embodiment of the present invention comprises a moldable soft plastic retainer 10 configured and dimensioned to be placed within the oral cavity O and snuggly fitted over the teeth T. Retainer 10 according to this exemplary embodiment includes plastic body 12 that serves as the platform and anchoring means to the teeth T, with embedded detector units 14 disposed thereon. Two detector units 14 are shown in this example, but persons of skill in the art will appreciate that any suitable number of detector units 14 may be included. Plastic body 12 may be formed by conventional techniques for fitting and manufacturing such retainers as is well understood in the dental arts. Detector units 14 may be affixed to the surface of plastic body 12 or in suitably sized cavities so as to present a relatively smooth outer surface. Fixation may be accomplished, for example, by the use of known dental adhesives. The adhesive and fixation technique selected by a person of ordinary skill in the art may be permanent or semi-permanent depending on whether or not the detector unit is to be replaced and/or reset as otherwise described herein. In a further alternative, detector units may be inserted into appropriately sized pockets formed in plastic body 12 by the user or healthcare professional. Also illustrated in FIG. 1 is optional additional detector 16, which may be visible only to the healthcare provider or the user when the retainer 10 is removed. Detector 16 may be secured in the same manner as detector units 14.

In one alternative embodiment, plastic body 12 is partly or wholly comprised of or includes a material that also provides the function of detector units 14 (and/or detector 16) to detect the substance of interest. In such embodiments, plastic body 12 serves as the detector unit as well as the platform and anchor means.

FIG. 2 illustrates another exemplary embodiment based on a retainer-type structure. In this embodiment, retainer 20 is a more traditional wire-type retainer including wire 22 that is specifically configured and dimensioned for close fit and retention on the user's teeth T. Such wire retainers are well known in the art and readily custom-formed for patients by dental care professionals. Wires 22 connect into a metal substrate 24, such as stainless steel, that is closely fitted to the roof R of the mouth and forms the platform for the detector unit(s). In this example, detector unit 14 as described above may be suitably permanently or semi-permanently bonded to substrate 24 as previously described. In a further alternative embodiment, rather than an adhesive fixation, detector unit 14 may be secured to substrate 24 by suitable clip means or other secure but readily removable means such that it could be readily replaced by the user as appropriate for a particular therapeutic regime. To increase fixation security, bands 26 may be optionally included. Bands 26 may be formed as are orthodontic bands known in the art. Alternatively, bands 26 may directly secure the platform, without a retainer device.

Retainer-based embodiments, such as exemplified in FIGS. 1 and 2, may be provided in several sizes that could then be form fitted by the user to closely approximate and affix to the upper and/or lower dentition. With plastic retainer bodies, such as used with retainer 10, hot water or other means could be used to temporarily make the material more pliable and thus custom fit to the dentition. As explained above, in various embodiments depending on the particular intended use or therapeutic program, one or more detectors may be semi-permanently or permanently affixed to the surface of the moldable soft retainer or inserted within the retainer. This would enable exchange of the detectors as needed, while retaining the same retainer as platform and anchor structure.

Further alternative embodiments of the present invention, as illustrated in FIGS. 3-8B, may be configured to be placed in the oral cavity exclusively by a healthcare professional such as a dentist, a physician, a dental technician or other expert skilled in the procedure of affixing dental devices. In this case, the device may be semi-permanently attached to one or more locations in the oral cavity thus preventing the user from intermittently removing the device. This would ensure that the device would remain in the oral cavity for 100% of the monitoring period.

Turning to FIG. 3, one such exemplary embodiment is described in more detail. As shown therein, a cavity C is provided in the face of tooth T. Detector 30 includes metal substrate 32, which carries detector unit 34. Detector unit 34 may comprise a suitable porcelain matrix impregnated with appropriate compounds as described in more detail below for detection of substances of interest. In one exemplary embodiment, the materials comprising detector unit 34 are appropriately fused into metal substrate 32 to facilitate handling, storage and implantation in the patient's teeth. In such an embodiment, detector 30 may be fixed within cavity C by bonding metal substrate 32 to the underlying tooth structure using permanent or semi-permanent dental adhesives as are well known to those skilled in the dental arts. Alternatively, the matrix comprising detector unit 34 may be directly bonded into cavity C by suitable dental adhesives or applied in a soft state and cured in place by conventional dental restoration techniques.

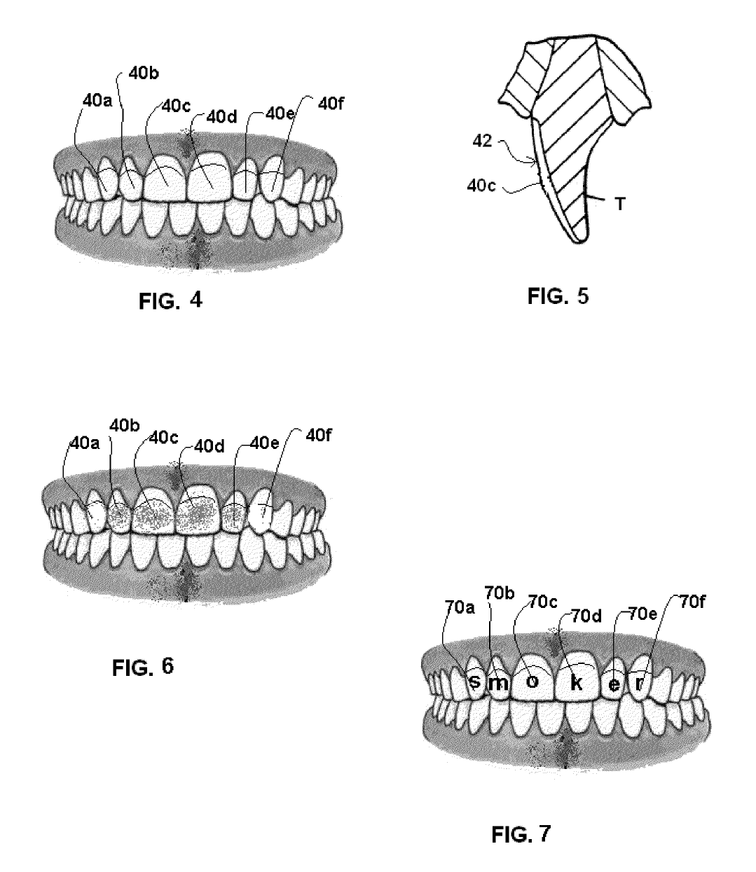

In further exemplary embodiments of the present invention, one or more bone screw anchors may be placed in the maxilla and/or mandible, upon which are disposed locking platforms that lock onto the screws. Detector units may be disposed on the platform or may be incorporated in the platform itself. The detector units may also be incorporated directly into the anchor system, as with a dental veneer. In this type of embodiment, the detectors may only be changed by the healthcare professional as needed for testing and renewal of the material. In another exemplary embodiment, as described in more detail below and shown in FIGS. 4-7, the anchor, platform and detector unit is incorporated as a single unit and configured as a dental veneer which is custom fit to one or more dental surfaces. In one example, dental veneers with innate detector capability could be applied to the buccal surface of the maxillary and/or mandibular dentition.

Each detector unit may be configured to detect one or more substances of interest as is appropriate for a particularly devised treatment regimen. If only one substance of interest is targeted (for example carbon monoxide to detect smoking behavior) using one or more detectors specific to that substance could be used. Alternatively, if more than one substance of interest is targeted (for example carbon monoxide to detect smoking behavior and ethyl alcohol to detect alcohol consumption), then multiple detectors each sensitive to only one substance could be used or one detector sensitive to multiple substance could be used.

There are various mechanisms by which the detectors detect a substance of interest. In one exemplary embodiment, the detector units present a color change when exposed to a substance of interest. Such color change could occur upon exposure to any level of the substance or to a specific pre-set threshold. Such color change could be reversible or irreversible. In another embodiment, the detector would be removed after a certain period of time and the color change detected and/or quantified by a healthcare provider. A new detector would be installed at each test visit for refreshed monitoring over the next period of time.

In one exemplary embodiment of a color-change based detector, carbon monoxide may be detected by materials wherein exposure to carbon dioxide induces color change. Such materials are known in the art; for example synthetic hemoglobin utilized in biomimetic CO sensors, and a number of other materials such as disclosed in U.S. Pat. No. 5,618,493, entitled Photon Absorbing Bioderived Organometallic Carbon Monoxide Sensors, which is incorporated by reference herein in its entirety.

In other exemplary embodiments, available colorimetric sensor materials may be employed to indicate the presence of other markers for other undesired behaviors. For example U.S. Pat. No. 4,900,666, which is incorporated herein by reference in its entirety, discloses a Colorimetric Ethanol Analysis Method and Test Device, including a test material that may be readily adapted for use as an alcohol sensor in an embodiment of the present invention. Known materials that provide colorimetric tests for cyanide (associated with cigarette smoking), such as pyridine-pyrazolone, may be used as sensors for many different smoked compounds that contain some amount of cyanide when burned. Colorimetric strip-based test materials also exist for compounds such as nicotine, arsenic, THC and cocaine, which also may be readily adapted for use in the present invention by persons of ordinary skill in the art.

Known detector kits for a variety of reagents also may be adapted for use with embodiments of the present invention by persons of ordinary skill in the art based on the teachings contained herein. Examples of such kits are disclosed in, for example, U.S. Pat. No. 5,063,164, entitled Biomimetic Sensor that Simulates Human Response to Airborne Toxins, U.S. Pat. No. 5,728,350 entitled Chemical or Microbiological Test Kit, U.S. Pat. No. 5,035,860, entitled Detection Strip for Detecting and Identifying Chemical Air Contaminants and Portable Detection Kit Comprising Said Strips, and US Patent Application Pub. No. 2009/0253220, entitled Absorbing Biomolecules into Gel-Shell Beads, each of which is incorporated by reference herein in its entirety.

Sensor materials as described above may be incorporated into detector units compatible with the oral environment in a number of ways as may be derived by persons of ordinary skill in the art based on the teachings contained herein. For example, the sensor material may be protected from the oral environment by a gas permeable, liquid impermeable barrier to permit gasses of interest that are present within the oral cavity to contact the sensor while isolating it from liquids in the oral cavity. Such gas permeable, liquid impermeable materials suitable for barrier membrane for protecting sensor materials according to the present invention are well known in the art. Examples of such materials are disclosed in U.S. Pat. No. 3,953,566, entitled Process for Producing Porous Products, and U.S. Pat. No. 4,904,520 entitled Gas-Permeable, Liquid-Impermeable Nonwoven Material, each of which is incorporated by reference herein in its entirety. Persons of ordinary skill in the art will be able to select other suitable barrier materials in addition to those described in the incorporated patents. However, sensors that work based on liquid contact may not require such protection. Alternatively, a porous porcelain substrate may be impregnated with sensor material to form a solid matrix that is biocompatible and safe for placement in the oral cavity while presenting an outer surface that is responsive to the presence of carbon monoxide. By forming a porcelain matrix such detector units may be secured to teeth, bone or substrate platforms using conventional dental materials, adhesives and processes well understood by persons in the art.

In the embodiments described in the preceding paragraph, the color change in the detector units may be configured to be irreversible. In an alternative exemplary embodiment, the detector unit may be constructed to provide a reversible color change when exposed to a substance of interest. Such reversible detectors may be made from synthetic hemoglobin to provide a reversible colorimetric sensor for the presence of carbon monoxide as is well known in the art.

With reversibly-colorimetric detector units, the material may change color upon a certain amount of smoke exposure, but will then slowly revert to normal color when exposure to smoke is decreased or avoided for a predetermined period of time. In this case, the user will have constant feedback about the amount of smoking that they are engaging in and will be constantly reminded that they are “over their limit”. Such a constant reminder will allow the user to immediately modify their behavior so as to allow the sensors to return to the normal color. In the case of irreversible colorimetric detectors, variable sensitivity of the material to carbon monoxide will allow tailoring of the monitoring and feedback for each patient. For example, in early phases of a smoking cessation program, the permanent colorimetric change may occur after smoking 100 cigarettes. With each exchange of the sensor units (weekly, for example), a more sensitive material is used, thereby allowing less and less smoking behavior before the colorimetric change occurs. By week eight, for example, the sensors change color after only ten cigarettes are smoked. By week twelve, the sensors change color after one cigarette is smoked.

In embodiments of the invention employing a reversible colorimetric detector unit, the variability in sensitivity to smoke exposure may be tailored according to the “time” and amount of exposure required to make a change, as well as the time with no smoke exposure required to return to normal. In one exemplary embodiment, the detector material, for example a hemoglobin disk, is a highly sensitive material that changes color relatively quickly upon exposure to cigarette smoke, and then requires a significant period of time, such as four to eight hours, or about six hours, of smoke-free (carbon monoxide free) environment before returning to normal.

In a further exemplary embodiment, detector units capable of quantifying longitudinal exposure to a substance of interest (e.g., carbon monoxide) may be employed alone or in combination with a visual detector such as a colorimetric detector unit as described above. This adjunctive monitoring device allows the healthcare professional to quantify exposure to carbon monoxide and therefore estimate smoking behavior during the monitoring interval. For example, the units may contain certain gel compounds, as described above, which absorb certain constituents of smoke over time or a passive computer chip capable of monitoring for carbon monoxide. When removed from the mouth and analyzed, the quantity of the smoke substance exposure can be compared to the time that the units were worn in the mouth, and a predictive algorithm used to estimate the number of cigarettes smoked during the entire time the units were worn (or converted to a cigarettes-per-day basis).

By utilizing quantitative detectors in conjunction with qualitative detectors, the patient would preferably be enrolled in an ongoing treatment program. For example, the patient would visit the smoking cessation program on a regular basis (i.e., weekly) at which time the detector units are removed and replaced with new ones. The old units are analyzed for quantitative evidence of smoking exposure according to time worn. This provides the healthcare provider with objective evidence of smoking behavior for the patient. Detector units used for quantifying exposure to a substance also need not be placed in visibly apparent portions of the oral cavity. Rather, they can be placed in discrete regions, such as on platform 24 of retainer 20 in FIG. 2, detector 16 in FIG. 1, or on a buccal surface of the molars when using an embodiment such as detector 30 in FIG. 3. The data is shared with the user at each visit and counseling and drug therapy altered accordingly.

In one exemplary embodiment of the present invention, as illustrated in FIGS. 4 and 5, detectors 40a-f are affixed to the buccal surface of the maxillary incisors and canine teeth. This may be accomplished, for example, as illustrated in FIGS. 4 and 5, by the use of porcelain tooth veneers as are known in the art. Detectors 40a-f may include any one or combination of the detector units described herein. In one exemplary embodiment, such porcelain veneers comprising integrated detector units as described herein are affixed to the teeth using known dental veneer procedures. One exemplary such procedure is disclosed in U.S. Pat. No. 4,992,049, the disclosure of which is incorporated by reference herein in its entirety. Optionally, a gas-permeable protective membrane 42, as illustrated in FIG. 5, may be employed as described above.

Thus, in the exemplary embodiment of FIGS. 4 and 5, the patient has six detectors 40a-f affixed to the upper dentition. The incisor detectors 40b-e are colorimetric detectors for carbon monoxide, while the canine detectors are able to quantify longitudinal exposure to carbon monoxide when removed and tested and replaced at periodic intervals. In this example, the patient is utilizing this device as part of a smoking cessation program. During normal daily activity, these detectors are visible to the user when looking in a mirror and are visible to user's acquaintances. At baseline, the colorimetric detectors may be clear or white and therefore indiscernible from normal braces or normal dentition. As such, the devices would be unobtrusive to both the user as well as individuals interacting with the user. As the user engages in smoking activity, the color of the material changes to an undesirable and/or unnatural color, such as brown, black, green, blue, etc., as shown in FIG. 6. (Note that because in this exemplary embodiment, detectors 40a and 40f are quantitative units, the color change is not presented on those teeth.)

If the detector material is irreversibly colorimetric, an option at each smoking-cessation visit is to remove the detectors and replace them with fresh detectors that have not been exposed to smoke. In this irreversible colorimetric embodiment, the user begins with a clean set of detector units at each visit and is reinvigorated and re-motivated to avoid smoking to avoid future undesirable color changes. If the user knows that they will be changing the units at some regular interval, even if they fail in one week, they can restart the clock with new units and try again. This reward/penalty system may improve compliance with the smoking cessation program.

When using colorimetric detectors as described herein, the change in color may involve the entire surface of the detector or it may be limited to certain portions of the detector, so as to spell out words or create images when activated, as illustrated by detector units 70a-f in FIG. 7.

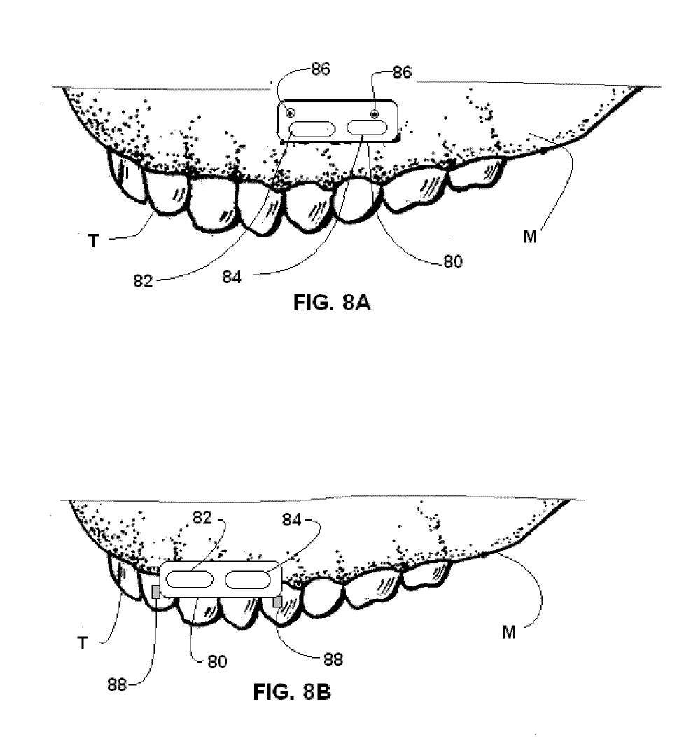

In a further alternative embodiment, as shown in FIG. 8A, detector 80, including a platform comprising a suitable biocompatible material such as titanium, stainless steel or various plastics, has disposed thereon detector units 82 and 84. As described hereinabove, detector units may be configured to test for the same or different marker substances. For example, detector units 82 and 84 may be comprised of a material such as synthetic hemoglobin that changes color when exposed to carbon monoxide and the other a sensor material that changes color in the presence of alcohol. Detector 80 is secured in place in this alternative using bone screws 86, placed into the bone, in this case the maxilla M. Preferably the detector is positioned sufficiently cranially so that bone screws 86 do not interfere with the teeth roots. When mounted on the mandible, the detector should be positioned sufficiently caudally for the same reasons. In the alternative embodiment of FIG. 8B, the same detector 80 is instead secured to teeth T with two brackets 88 that are adhered to the teeth with known bonding techniques as typically used in orthodontics. The platform may be secured to brackets 88, for example, by clips if a removable attachment is desired or more permanently fixed by wires or adhesive.

The various alternative and exemplary embodiments described herein present methods and devices that are well adapted to and may be used in conjunction with a formal behavioral cessation program, such as a smoking, drug, or alcohol cessation program staffed by healthcare professionals and in which drug and counseling therapies are utilized. In such cases where the patient is participating in a fully-supported program, these methods and devices would serve as adjuncts to currently accepted therapies in that they would provide detection of undesirable behavior during unmonitored time periods. This would provide the healthcare professional with additional information not normally available regarding patient compliance during unmonitored intervals. Such additional information would allow the healthcare provider to adjust therapy according to patient response, thus optimizing therapy and increasing efficacy. Such additional information allows more optimized care and tailoring of therapy. These methods and devices would provide positive and negative feedback to the user, and accountability for them to adhere to their program.

Alternatively, these methods and devices may be used independently by a patient who is not participating in a formal behavioral modification program. Such a patient may be self-motivated and may wish to have additional incentive or assistance in quitting the specific undesirable behavior, without outside assistance. These methods and devices would provide positive and negative feedback to the user, and accountability for them to adhere to their program.

Alternatively, these methods and devices may be used after completion of a cessation program once the behavior has been purportedly extinguished. In this use, the monitoring can provide objective evidence to confirm that the behavior has been extinguished and can provide long-term monitoring to increase the likelihood of maintaining abstinence (avoiding recidivism).

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

What is claimed is:

1. An apparatus for detecting substances within a patient's oral cavity as markers for undesired behaviors, comprising:
a platform configured to be secured within the oral cavity of a patient; and
at least one passive detector unit disposed on the platform and adapted to be disposed in the oral cavity, said detector unit capable of detecting one or more substances indicative of the undesirable behavior.

2. The apparatus of claim 1, wherein the detector is capable of detecting one or more substances of interest within inhaled gas and particulates, exhaled gas and particulates, intra-oral gasses, ingested liquids and solids, intra-oral mucous, secreted saliva, and other means.

3. The apparatus of claim 2, wherein the detector is capable of detecting intra-oral substances indicative of smoking, alcohol or drug use.

4. The apparatus of claim 3, wherein said detector is capable of detecting carbon monoxide.

5. The apparatus of claim 3, wherein said detector is capable of detecting nicotine.

6. The apparatus of claim 3, wherein said detector is capable of detecting THC.

7. The apparatus of claim 3, wherein said detector is capable of detecting cocaine.

8. The apparatus of claim 3, wherein said detector is capable of detecting cyanide.

9. The apparatus of claim 3, wherein said detector is capable of detecting arsenic.

10. The apparatus of claim 1, further comprising at least one anchor element configured and dimensioned to secure the detector unit and platform within the oral cavity.

11. The apparatus of claim 10, wherein said anchor element comprises at least one bone screw configured for insertion into at least one of the maxilla or mandible.

12. The apparatus of claim 10, wherein the anchor element comprises an adhesive.

13. The apparatus of claim 10, wherein the anchor element comprises an orthodontic band, bracket, or retainer configured to be affixed to the teeth by orthodontic means.

14. The apparatus of claim 10, wherein the anchor element comprises a retainer molded to fit over the dentition.

15. The apparatus of claim 10, wherein the anchor, detector, and platform are distinct components.

16. The apparatus of claim 10, wherein the anchor, detector and platform are combined in an integrated component.

17. The apparatus of claim 1, wherein the anchor and platform comprise the detector, an example of which is a dental veneer that changes color, or a clear retainer that changes color.

18. The apparatus of claim 1, wherein the detector unit is covered by a gas permeable, liquid impermeable barrier.

19. The apparatus of claim 1, wherein the platform comprises a porous material and the detector unit comprises a material contained within pores of said porous material.

20. The apparatus of claim 1, wherein the platform comprises a dental veneer configured to be adhered to a tooth surface.

21. The apparatus of claim 1, wherein the platform comprises a porcelain inlay configured to be installed within a tooth structure.

22. The apparatus of claim 1, wherein the detector unit comprises a material that changes color upon exposure to a substance of interest.

23. The apparatus of claim 21, wherein the color change is irreversible.

24. The apparatus of claim 21, wherein the color change is reversible.

25. The apparatus of claim 1, wherein the detector is removable for analysis and is capable of quantifying the amount of substance exposure over time when removed and analyzed.

26. The apparatus of claim 1, wherein the apparatus is configured and dimensioned to be permanently or semi-permanently installed in the oral cavity by a healthcare professional and is configured to prevent the user from removing the apparatus or components of the apparatus.

27. The apparatus of claim 1, wherein the apparatus is configured and dimensioned to be semi-permanently installed in the oral cavity by a healthcare provider and is configured to allow either the patient or healthcare provider to exchange at least the detector unit.

28. The apparatus of claim 1, wherein the apparatus is configured and dimensioned for placement in and removal from the oral cavity by the patient.

29. An apparatus for detecting substances within a patient's oral cavity as markers for undesired behaviors, comprising:
a passive detector unit comprising a material that changes color when exposed to a selected marker substance; and
a platform for supporting and securing the passive detector unit within the oral cavity.

30. The apparatus of claim 29, wherein said material comprises hemoglobin.

31. The apparatus of claim 29, wherein said platform is configured as a retainer and said detector unit material is disposed within compartments formed in said retainer.

32. The apparatus of claim 31, wherein said compartments are separated from the oral cavity by a gas permeable, liquid impermeable barrier.

33. The apparatus of claim 29, wherein said platform comprises a dental material adapted to be affixed to at least one of the patient's teeth.

34. The apparatus of claim 33, wherein the platform comprises a dental veneer.

35. The apparatus of claim 33, wherein the platform comprises a porcelain inlay.

36. An apparatus for detecting substances within a patient's oral cavity as markers for undesired behaviors, comprising a substrate of dental material adapted to be affixed to at least one of the patient's teeth, and a composition of hemoglobin disposed on said substrate such that exposure to carbon monoxide through smoking causes visible discoloration of the patient's teeth.

37. The apparatus of claim 36, wherein the hemoglobin is isolated from liquids in the patient's oral cavity by a gas permeable, liquid impermeable barrier.

38. The apparatus of claim 36, wherein the hemoglobin is configured to appear as letters or symbols when exposed to carbon monoxide.

39. A method for revealing participation in undesired behavior, comprising:
passively and continually monitoring for the presence of at least one marker substance within a patient's intra-oral space, the marker substance being a substance indicative of the undesired behavior; and
displaying a visual indicator within patient's intra-oral space when the marker substance is detected as present.

40. The method of claim 39, wherein said passively and continually monitoring comprises installing at least one passive detector within the intra-oral space.

41. The method of claim 40, wherein said at least one passive detector comprises a material that changes color upon exposure to said marker substance.

42. The method of claim 41, wherein the color change is reversible upon removal of exposure to said marker substance.

43. The method of claim 39, wherein said displaying includes positioning said detector in a location within the patient's intra-oral space that is outwardly visible to others.

44. The method of claim 43, wherein said displaying appears as a visible discoloration of at least one of the patient's teeth.

45. The method of claim 44, wherein said displaying comprises displaying letters or symbols within the patient's intra-oral space.

46. The method of claim 39, wherein said displaying includes positioning said detector in a location visible only to the patient or other person upon inspection of said detector.

47. The method of claim 40, wherein said installing comprises affixing said at least one detector to at a least one patient tooth.

48. The method of claim 40, wherein said installing comprises placing a retainer in the patient's mouth.

49. The method of claim 39, wherein said monitoring and displaying comprises a part of a formal behavior modification program.

50. The method of claim 49, wherein the formal behavior modification program is a smoking-cessation program or an alcohol-dependency program.

51. The method of claim 39, wherein said monitoring and displaying comprise a part of a patient self-motivated behavior modification program.

52. The method of claim 39, further comprising passively and continually monitoring for more than one said marker substance by installing multiple substance detectors within the intra-oral space.
